(19) United States
(12) Patent Application Publication
(10) Pub. No.: US 2003/0079990 A1
(43) Pub. Date: May 1, 2003

(54) Method of electro-coating an article

(76) Inventors: Wang Xiangdong, Singapore (SG); Loh Lee Foon, Singapore (SG); Fang Shunong, Singapore (SG)

Correspondence Address: HARNESS, DICKEY & PIERCE, P.L.C., P.O. BOX 828, BLOOMFIELD HILLS, MI, 48303, US

(21) Appl. No.: 10/236,066

(22) Filed: Sep. 5, 2002

(30) Foreign Application Priority Data
Sep. 6, 2001 (SG) 200105417-0

(51) Int. Cl. C25D 1/12; G01L 1/20; C07K 1/26; C02F 1/469; G01F 1/64; C08F 2/58

(52) U.S. Cl. 204/478; 204/491

(57) ABSTRACT

A method for coating an article with a electro-composite coating includes supplying a resin having a particulate material suspended therein to a main vessel from a secondary vessel, said secondary vessel having a stirring means for maintaining the particular material in suspension, immersing at least the surface of the article to be coated in the main vessel and conducting electricity from a DC source through the solution and the article to coat the solution and suspended particles onto the outer surface of the article removing the article from the main vessel and removing access coating materials from the article, and curing the coating onto the article.

### FIELD OF THE INVENTION

This invention relates to a method of an apparatus for coating articles by electrophoresis.

### BACKGROUND OF THE INVENTION

In the production of coating layers by electrophoresis, macro-molecules are transported under the effect of an electric field onto a conducting body acting as an electrode. Generally the primary objective of an electro-coating process is to produce a smooth homogeneous coating surface on the substrate electrode. However the applicant's intention is to produce an article which has a roughened coated surface to increase the contact friction between the coated article and objects which are contacted by the article.

### SUMMARY OF THE INVENTION

To achieve this, the applicant's objective of the invention is to provide a method of coating an article with a composite coating layer in which the coated article has an roughened surface.

Accordingly, in the broadest form the invention provides a method of applying a composite electro-coating to an article comprising the steps of:

contacting the article with an electrophoresis resin solution containing a particulate material suspended therein, the article being connected to a source of DC current,

conducting electricity through the article and the solution,

removing the article from the solution, and

curing the coating layer on the article.

In another aspect of the invention, there is provided a method for coating an article with a composite coating comprising the steps of:

supplying a resin having a particulate material suspended therein to a main vessel from a secondary vessel, said secondary vessel having a means for suspending the particulate material in suspension in an electro-coating solution,

immersing at least the surface of the article to be coated in the main vessel and conducting electricity from a DC source through the solution and the article to coat the solution and suspended particles onto the outer surface of the article,

removing the article from the main vessel and removing excess coating materials from the article, and curing the coating onto the article.

In order to provide a substantially uniformed thickness across the coating surface, it is preferable that the process variables such as temperature, pH and particle content and size are controlled. The controlled ranges of these variables will depend on the resin used.

The electrophoresis resin solution may be formed from a suitable electrophoresis resin or resins, solvent and other film forming materials including binding agents and extenders. The electrophoresis resin is preferable a polyurethane resin but other organic coating materials such as alkyd resins, acrylic resins and/or epoxy resins or mixtures of the foregoing may be used.

Conductivity is a key factor for composite e-coating. The thickness (particle coverage density) is most effected by conductivity. Generally, the lower the conductivity, the higher the thickness. In a normal system, pH and particle concentration which may also affect thickness are more stable and more easily controlled than conductivity in a given period of time. Relatively, conductivity becomes the most sensitive factor which affects thickness. In fact, good control of conductivity is a highly preferred to attain good coating results. In most cases, unstable conductivity affects the variation of dimension among batches. Experientially, the variation of conductivity should be controlled within ±5 μS/cm.

It is preferable that the means of suspending the particulate material in the e-coating solution is a stirrer. The action of the suspension means causes bubbles to form in the solution. Since the conductivity is effected by the amount of air bubbles in the e-coating solution, the control of conductivity may be accomplished by controlling the air bubble content in the e-coating solution in the main vessel. This may be accomplished by controlling the stirring speed of the suspension means.

The bubbles formed in the solution and are entrained in the solution which passes to the main vessel have a direct effect on the conductivity of the e-coating solution which must be controlled in the main vessel in order to provide a uniform coating. Therefore, it is preferable to control the amount of bubbles entrained in the e-coating solution entering the main vessel. This may be accomplished by the suspended e-coating solution passing upwardly through the lower buffer region in the main vessel. The lower buffer region may be a diverging section preferably at the base of the main vessel.

It is preferable that the particulate material dispersed in the solution is at a concentration of between 50 to 500 g/l and that the particulate material is at least one of the materials selected from the group of alumina, silica and silicon carbide. It is preferable that these particles are in the range of 1 to 100 microns.

While the pH of the coating solution may controlled to be within the range of 4.1 to 4.6, the preferred range is 4.2 to 4.5. Prior to contacting the article with the resin solution the surface of the article preferably undergoes a pre-treatment step in which the surface of the article undergoes an alkaline and acid treatment.

It is also preferred that the built up of the coating is conducted at a lower voltage for an initial predetermined period of time followed by an increase in the voltage for a second predetermined period of time.

In a further aspect of the invention there is provided an apparatus for coating an article comprising:

a main e-coating vessel for receiving the article to be coated,

a secondary vessel having a means for suspending a particulate material in an e-coating solution, said suspension means creating air bubbles which are entrained with the suspended particulate material to the main vessel, means to convey the suspended particulate material to the main vessel, and means to convey the overflow solution from the main vessel to the secondary vessel, wherein the main vessel is oriented to allow the suspended particles to be carried towards the article to be coated.

Preferably the electro coating solution enters the main vessel through a lower buffer region below the region where the article is contacted with the solution. The lower buffer region may be a diverging section at the base of the main vessel.

Further features objects and advantages of the present invention will be become more apparent from the following description of the preferred embodiment and accompanying drawings.

### BRIEF DESCRIPTION OF THE DRAWINGS

FIG. 1 is a schematic diagram illustrating an apparatus for carrying out the method of the invention,

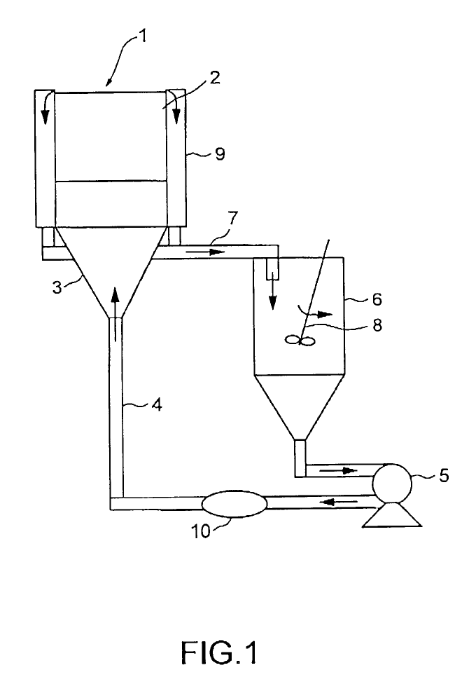

FIG. 2 is a graph illustrating particle content in the coating solution as a function of pH,

FIGS. 3(a)-(d) are graphs illustrating the change in thickness and conductivity of the applied coating with respect to alumina content at varying pH levels of (a) 4.1, (b) 4.2, (c)4.4 and (d) 4.3, and

FIG. 4 is a flow diagram illustrating the method of the invention.

### DESCRIPTION OF THE PREFERRED EMBODIMENT

The invention is suitable for use with electrophoresis or e-coating resins commercially available. Typical e-coating resins have an epoxy, acrylic, alkyd or polyurethane base with polyurethane being the most preferred. The preparation of these resins would be well known to those skilled in the art and does not form part of the invention.

Referring to the drawings, a typical apparatus for carrying out the method of the invention is shown comprising a main vessel 1 having an upper work zone 2 and a lower buffer zone 3. The resin solution enters the vessel generally through the bottom of the main vessel 1 into the buffer zone 3 from a conduit 4 connected to a pump 5.

Pump 5 draws resin with particulate material in suspension from a secondary overflow vessel 6 which receives resin solution from an overflow duct from the main vessel 1. The main vessel 1 is supplied with an overflow trough, preferably circumferentially around the perimeter thereof which flows into overflow conduit duct 7.

The secondary overflow vessel 6 may be positioned below the main vessel 1 so that the overflow resin from the main vessel 1 flows under the influence of gravity into the overflow vessel 6. The overflow vessel is provided with a means to maintain the particulate material in suspension such as a stirrer 8. While the operation of the stirrer is necessary to keep the particles in solution and thereby help to attain even coating and particle coverage, the operation of the stirrer generates bubbles in the overflow vessel. As the presence and the amount of bubbles effects the conductivity of the solution, this variable can be controlled by adjusting the stirrer speed. Generally, the higher the stirring speed, the more bubbles are generated and the lower the conductivity. When the stirring speed is adjusted, it may take hours for the conductivity in the system to stabilize.

The pump is preferably an air driven pump which is able to provide a fairly constant flow of solution through the conduit at a rate which depends on the size and residence time of the resin solution in the main vessel 1. The flow rate may typically be 20-80 liters/min for a residence time of between 0.5-5minutes. In order to further control the flow rate, a flow equalizer 10 is also provided in conduit 4.

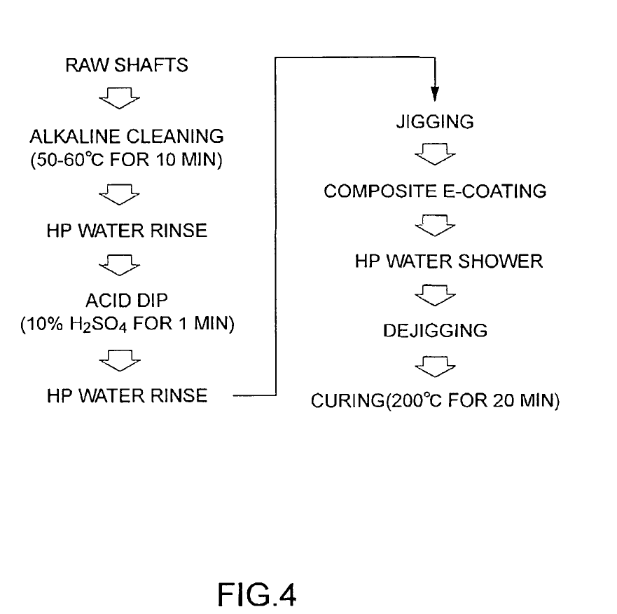

Referring to FIG. 4, a flow diagram of a preferred embodiment of the method of the invention. The method was performed on cylindrical shafts approximately 31.16 cm long and 12.115 cm in diameter. The shafts are made of carbon steel. Those sections of the shaft which are not to be coated are covered with for example end caps. The shafts then undergo a pretreatment which consists of an alkaline cleaning in an alkaline solution. The preferred alkaline pretreatment used in the examples is in a solution consisting of NaOH, Na₂CO₃, Na₃PO₄.12H₂O, Na₂SiO₃ made up to 140 g/l for a period of about 10 minutes at a temperature of about 60° C. The alkaline pretreatment is then followed by an acid wash in 10% H₂SO₄ solution. Pretreatment is highly desirable for composite e-coating before a shaft is coated. It includes alkaline cleaning and acid immersion. The purpose of pretreatment is to get a clean surface which can increase adhesion and avoid coating problems.

The pretreatment process is as follows:

Alkaline cleaning (60° C.) for 10 min→High Pressure water rinse→acid (10%H₂SO₄) immersion for 1 min→High Pressure water rinse→drying

The shafts are fitted to a jig or carrier which is lowered into composite electro-coating solution in the main vessel 1. Currents are then passed through the solution and the coating resin and particulate material is coated onto the metal substrate. The suitable temperature for composite e-coating is between 22-24° C. Higher temperatures can result in solution broken down. Although solution is more stable at low temperatures, a lower temperature will reduce the coating (resin) thickness.

The coated substrates are then subjected to a shower before de-jigging and passing to an oven or kiln to cure at a temperature of about 200° C. for approximately 20 minutes.

It has been discovered that the process parameters have an effect on the thickness and uniformity of the applied composite coating. The applicants have conducted further tests on the process parameters of the coating process to determine the preferred conditions for the process. In order to enable the process to operate satisfactorily, it is preferable that the process variables be maintained within specific ranges. The particles used were alumina having particle sizes of average diameter is 48.0+/−3.0 μm and average diameter is 40.0+/−2.5 μm in which the preferred average diameter to produce the required coating thickness was found to be in the size range of 48.0+/−3.0 μm.

The preferred resin used by the applicants is a polyurethane resin marketed by Hawking International (HK) under the trademark CLEARCLAD. Dye may be added to the resin. Suitable dyes include those also marketed by Hawking International (HK) Ltd. under the trademark CLEARCLAD. The preferred dye of the applicants during experiments was a mixture of the e-coating resin and carbon black in an addition rate of 100-200 ml/l polyurethane resin. A higher dye content will be of assistance to achieve better appearance, but it will reduce the particle coverage density.

A solvent is preferably used in the resin/particle solution. The solvent is preferably a mixture of a disperse phase solvent and a continuous phase solvent. Solvents are selected as appropriate for the e-coating resin used. For the polyurethane resin used by the applicants, solvents marketed by Hawking International (HK) Ltd for the CLEARCLAD system are A250 and A264.

When used for quite a long time without any monitoring and maintenance, the amount of solvent in solution will decrease due to evaporation and vaporization. Low solvent level will result in problems such as thin (resin) coating, uneven coating out and lumps etc. When the problems appear, solvent analysis should be done.

A250 is the disperse phase solvent. It is not miscible with water and can never be added to the bath alone. It coats out with the resin and promotes flow in the stove giving brightness to the coating. A264 is the continuous phase solvent. It is completely miscible with water and can be added directly to the bath. It helps formation of the coating and adds to the brightening effect of the A250 solvent.

A250 can be added to the bath provided it is mixed thoroughly with A264 in a ratio of 1:2 or more. Addition should be made slowly through the weir (overflow vessel) and at least 30 minutes should be allowed for mixing before using the bath (overnight is preferable).

At 10% solid content, solvent content A250 should be between 2.5-3.5% and A264 is between 3-6%. This is also the range used in our testing. Higher solvent level (out of this range) is not encouraged, because high solvent level will greatly increase the coating (resin) thickness and reduce the particle coverage density thereby increasing the chance of uneven appearance.

If solvents are lower than the recommended ranges, it will result in a thin resin coating and lumps. (a coating layer contains resin coating and particles). Lack of solvent, affects the formation of resin coating so increasing the percentage of particles in a coating layer. Too high percentage of particles may result in particle accumulation or lumps. Lumps reduce the contact areas so result in lower friction. Addition of solvent can promote flow and leveling in the stove and reduce the lumps, both of which may be of help to friction.

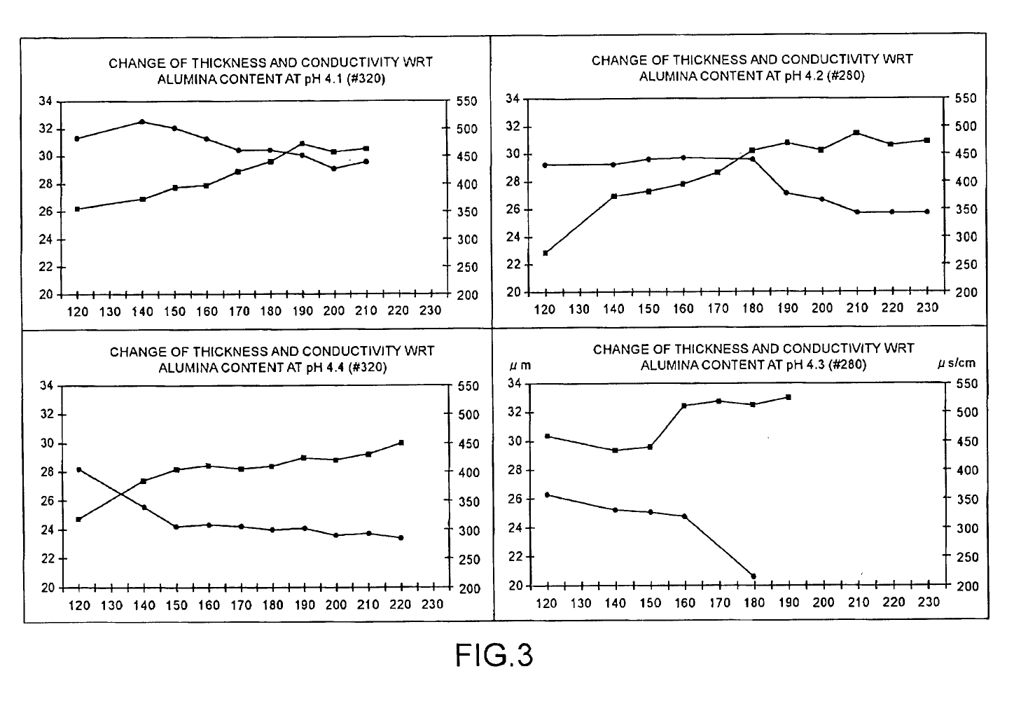

In order to produce coatings of a uniform thickness of say, 50 microns and even particle coverage, the particle concentration in the resin should not be less than 100 g/L. The minimum particle concentration to obtain a desirable thickness varies with the pH value of the solution (see table 1). However, a higher particle concentration in the solution will enable a coating of a desirable dimension to be achieved more easily. In practice, a particle concentration of 200 g/l-250 g/l is applied at a pH range of 4.2-4.5. The results of Table 1 are shown in FIG. 3.

Due to the presence of bubbles in the resin solution, the practical volume of solution in the system is hard to measure. Hence, the particle concentration is calculated as follows:

Particle concentration=the amount of particle added/the volume of solution made up

The applicants found it preferable to circulate solution and particles overnight to mix them even before starting a sample making. New make up solution must be circulated with particles (alumina) over 12 hours to achieve the stability. Stable circulation is of help to maintaining the system stable and a constant flow can reduce the variation of conductivity and coating thickness.

TABLE 1

| | Min. Particle Concentration, |
|---|---|
| pH | g/L |
| 4.10 | 190 |
| 4.20 | 180 |
| 4.30 | 160 |
| 4.40 | 150 |
| 4.55 | 120 |

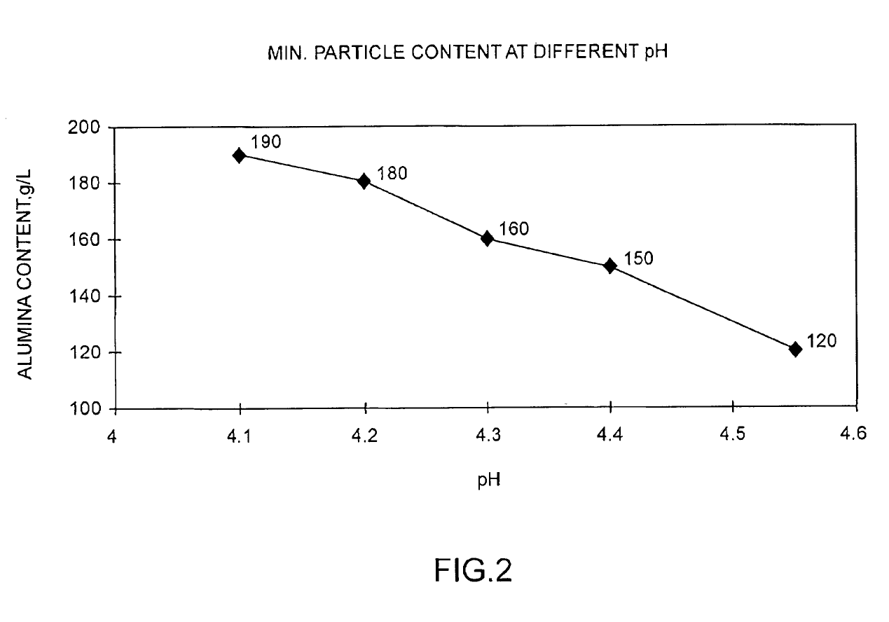

With particle concentration increasing, the conductivity of solution will decrease slightly. But when the particle concentration increases up to a certain point which varies with the pH value, the conductivity will have a sudden drop in a large scale (see FIG. 2). This point seems be right the minimum particle concentration to obtain even particle coverage.

As a result of the above trials the applicants consider that the coating process of the invention is best performed at a pH level of about 4.2-4.5. At pH=4.1 or below, the appearance of coating on the sample substrates is non-uniform and not good. However, when the pH is 4.6 or higher, non-uniform lumps may appear in the coating. It is recommended to control the solution between pH 4.2-4.5.

The pH value of a new makeup solution with 10% solid content and 200 ml/l dye has a pH of around 4.35 which is suitable for composite e-coating. The pH value may slightly vary with different solid contents and dye contents. (see table 2)

TABLE 2

| Solid | | |
|---|---|---|
| content | Dye | pH |
| 8% | / | 4.22 |
| 10% | / | 4.25 |
| 12% | / | 4.29 |
| 10% | 50 ml/L | 4.30 |
| 10% | 150 ml/L | 4.36 |
| 10% | 250 ml/L | 4.42 |

pH values will slightly increase (about 0.1 unit) with addition of alumina.

In the composite e-coating system of the invention, measurement and control of pH and conductivity of the solution are important. In order to monitor the effects of pH on the quality of the applied composite coating, a 200 m/l sample of solution was taken and allowed to stand for about an hour to allow the solids to settle and the bubbles to dissipate. The pH is then measured using a clean calibrated pH meter.

The applicants have found that if a solution is used for too long a period of time or after a large production run without any maintenance, the pH of the solution will tend to increase. An increasing pH may result in solution broken down, resulting in such problems as uneven coating out, lumps etc. A pH value of 4.8 was measured in a broken down solution. The applicants are of the view that a pH level which is too high or too low results in an uneven coating and a poor appearance. Frequent measuring (at least once a day) is necessary to keep the solution in a stable state.

Generally, a decreasing pH solution has not been found to occur during normal operations. If the pH of solution is found to be becoming a little too high (such as pH up to 4.6), the solution should be monitored more frequently. In some cases, it is recommended that a small amount of ES (Emulsion Stabilizer) should be added into solution to reduce pH. ES can greatly reduce pH and increase the conductivity of solution, so the addition of ES must be done carefully. 0.01%-0.02% volume of ES is a suitable amount to added into solution at no shorter frequency than every 15 minutes.

Conductivity is a key factor for composite e-coating. The thickness (particle coverage density) is most effected by conductivity. Generally, the lower the conductivity, the higher the thickness. In a normal system, pH and particle concentration which may also affect thickness are more stable and more easily controlled than conductivity in a given period of time. Relatively, conductivity becomes the most sensitive factor which affects thickness. In fact, good control of conductivity is a highly preferred to attain good coating results. In most cases, unstable conductivity affects the variation of dimension among batches. Experientially, the variation of conductivity should be controlled within ±5 μS/cm.

The static conductivity of solution is between 400-500 μS/cm (10% solid content,50-250ml/l, see table 3). This value is much greater than that measured in an actual circulating system. This is mainly due to the particles and bubbles in an actual circulating system. The conductivity may reduce by 50 μs/cm when particles are added, and bubbles which are mostly produced by stirring may greatly reduce conductivity by at least 200 μS/cm.

The actual conductivity is more useful than the static one. It is of no significance to measure the static conductivity for sample making. The static conductivity in system/solution is nearly always constant. But the conductivity measured in an actual circulating system is not constant. It can be or only be affected by the bubbles if the particle concentration, solid content and dye content are fixed. The testing results show that it is the actual conductivity and not the static conductivity which effects thickness. The lower the actual conductivity, the higher the particle coverage density, resulting in a higher coating thickness. Furthermore a lower actual conductivity was found to help to achieve a more even particle coverage.

TABLE 3

| Solid | | Conductivity |
|---|---|---|
| content | Dye | (μS/cm) |
| 8% | / | 416 |
| 10% | / | 443 |
| 12% | / | 489 |
| 10% | 50 ml/L, | 445 |
| 10% | 150 | 389 |
| 10% | 250 | 395 |

Conductivity should be measured frequently to keep the variation of conductivity within a small range. However, if the conditions (particle concentration, solid content, dye content, stirring speed, flow rate and the volume of solution etc.) are kept fixed, the conductivity will also remain constant.

During trials, good substrate composite coating was attained at a conductivity range of 260-340 μS/cm, where the particle concentration is 200-250 g/l. Good coatings were also attained at higher conductivity (400-440 μS/cm) when particle concentration was up to 300 g/l.

Conductivity can be adjusted by adjusting the stirring speed. Generally, the higher the stirring speed, the lower the conductivity.

During trials, good particle coverage was obtained between 7%-12% solid content (dye 50-250 ml/L). It was found that at a solids content of 6%, samples with desirable particle coverage could not be achieved. But higher solid content slightly reduce the particle coverage density if the same particle concentration is applied. So 10±1% solid content was found to be the most suitable.

In a preferred form of the invention, a two-voltage method is used in which 15 volts is applied for 30 seconds then 40 volts for another 30 seconds (15V30S40V30S). A two-voltage application method has been found to be more effective than one-voltage to control thickness. The particle coverage density always determines the thickness of the coating. By the two-voltage method, you can reduce the first voltage to increase the particle coverage density. If the second voltage is raised, percentage of coating (resin) increases, which results in decreasing of particle coverage density. But thickness can only be adjusted in a limited range at a given condition.

In a system for composite electro-coating of the cylindrical rods as described earlier containing polyurethane resin and alumina with a particle size range of 48.0 μm. The most favourable conditions for attaining a high quality coating are as follows:

| | |
|---|---|
| Solid content | 9-10% |
| Dye | 150-200 ml/l |
| Particle concentration | 200-250 g/L |
| pH | 4.2-4.5 |
| Conductivity | 260-340 μS/cm |
| Temperature | 22-24° C. |
| Solvent A250 | 2.5-3.5% |
| Solvent A264 | 3-6% |
| Voltage and Time | 15V30S40V30S |
| Curing | 200° C. for 20 min |

The trial substrates were a series of 18 elongate cylindrical rods 31.16 cm in diameter labeled A to R. The average coating thickness for each rod in the batch as measured by laser micrometer is recorded in Table 4. The variation in coating thickness within rods in the sample is shown in Table 5.

As can be seen from the results the average thickness of the coating of the sample rods varied by a maximum of 2 μm. The maximum variation within each rod was also 2 μm.

The method according to the invention enables a composite coating of resin and particulate matter to be applied to a substrate. By carefully controlling the process parameters, the resulting coating is produced with a substantially uniform thickness and particle coverage.

The entire disclosure of Singapore Patent Application No. 200105417-0 filed Sep. 6, 2001 is hereby incorporated by reference.

1. A method of applying a composite electro-coating to an article comprising the steps of:
contacting the article with an electrophoresis resin solution containing particulate material suspended therein, the article being connected to a source of DC current,
conducting electricity through the article and the solution thereby forming a coating layer,
removing the article from the solution, and
curing the coating layer on the article.

2. The method of claim 1, wherein the particulate material is suspended in a secondary vessel, the suspended particulate material passing to a main vessel for contact with the article.

3. The method of claim 2, wherein the solution contains bubbles from the secondary vessel which is fed into a lower region of the main vessel before contact with the article to be coated.

4. The method of claim 3, wherein at least one of the process variables selected from the group of temperature, pH, particle content and size are controlled to provide a coating layer of substantially uniform thickness and coverage.

5. The method of claim 4 wherein the resin is an organic resin selected from the group of acrylic resins, alkyd resin, epoxy resin and polyurethane resin and/or mixtures of the foregoing or other film forming materials including binding agents and extenders.

6. The method of claim 5, wherein the resin is a polyurethane resin.

7. The method of claim 4, wherein the particulate material is at least one of the materials selected from the group of alumina, silica and silicon carbide.

8. The method of claim 4, wherein the particulate material dispersed in the resin is at a concentration of between 50 to 500 g/l.

9. The method of claim 4, wherein the particle size diameter of the particulate material is less than 100 μm.

10. The method of claim 4, wherein the pH of the coating solution is controlled to be within the range of 4.1 to 4.6.

11. The method of claim 10, wherein the pH is controlled to be within the range of 4.2 to 4.5.

12. The method of claim 11, wherein prior to contacting the article with the resin solution the surface of the article undergoes a pre-treatment step in which the surface of the article is subjected an alkaline treatment followed by an acid treatment.

13. The method of claim 12, wherein the built up of the coating layer is conducted at a lower voltage for an initial predetermined period of time followed by an increase in the voltage for a second predetermined period of time.

14. An electro-coating method for applying a composite coating comprising the steps of:
supplying a resin having a particulate material suspended therein to a main vessel from a secondary vessel, said secondary vessel having a means for suspending the particulate material in an electro-coating solution,
immersing at least the surface of the article to be coated in the main vessel and conducting electricity from a DC source through the solution and the article to coat the solution and suspended particles as a coating onto the outer surface of the article,
removing the article from the main vessel and removing excess coating materials from the article, and
curing the coating onto the article.

15. The method of claim 14, further including the step of controlling at least one of the process variables selected from the group of conductivity, temperature, pH, particle content and size to provide a coating of substantially uniform thickness and particle coverage.

16. The method of claim 14, wherein the built up of the coating is conducted at a lower voltage for an initial predetermined period of time followed by an increase in the voltage for a second predetermined period of time.

17. The method of claim 16 wherein the resin is an organic resin selected from the group of acrylic resins, alkyd resin, epoxy resin and polyurethane resin and/or mixtures of the foregoing or other film forming materials including binding agents and extenders.

18. The method of claim 17, wherein the resin is a polyurethane resin.

19. The method of claim 14, wherein the particulate material is at least one of the materials selected from the group of alumina, silica and silicon carbide.

20. The method of claim 15, wherein the particulate material dispersed in the resin is at a concentration of between 50 to 500 g/l.

21. The method of claim 14, wherein the particle size diameter of the particulate material is less than 100 μm.

22. The method of claim 14, wherein the pH of the coating solution is controlled to be within the range of 4.1 to 4.6.

23. The method of claim 22, wherein the pH is controlled to be within the range of 4.2 to 4.5.

24. The method of claim 23, wherein prior to contacting the article with the resin solution the surface of the article undergoes a pre-treatment step in which the surface of the article is subjected an alkaline treatment followed by an acid treatment.

25. An apparatus for coating an article comprising:
a main coating vessel for receiving the article to be coated,
a secondary vessel having a means for suspending a particulate material in an coating solution, said suspension means creating air bubbles which are in the solution with the suspended particulate material,
means to convey the solution to the main vessel, and means to recapture and return overflow solution from the main vessel to the secondary vessel, wherein the main vessel is oriented to allow the suspended particles to be carried towards the article to be coated.

26. The apparatus of claim 25, wherein the coating solution enters the main vessel through a lower buffer region below the region where the article is contacted with the solution.

27. A method of applying a composite electro-coating to an article comprising the steps of:
mixing particulate material in a secondary vessel to form an electrophoresis solution;
feeding the solution to a primary vessel containing the article;
conducting electricity through the article and the solution thereby forming a coating layer;
removing the article from the solution; and
curing the coating layer on the article.

28. The method of claim 27 which further comprises:
feeding the solution into a lower portion of the primary vessel;
collecting solution that overflows from the primary vessel; and
returning the overflow solution to the secondary vessel.

29. The method of claim 28 which further comprises:
stirring the solution in the secondary vessel to create bubbles in the solution.

30. The method of claim 29 which further comprises:
increasing the voltage of the electricity to a higher level after a predetermined amount of time.
